(19) United States
(12) Patent Application Publication
(10) Pub. No.: US 2007/0189918 A1
(43) Pub. Date: Aug. 16, 2007

(54) Device and method for generating excited and/or ionized particles in a plasma

(75) Inventor: Alexander Gschwandtner, Munchen (DE)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: R3T GmbH Rapid Reactive Radicals Technology

(21) Appl. No.: 11/704,978

(22) Filed: Feb. 12, 2007

(30) Foreign Application Priority Data
Feb. 10, 2006 (DE) 10 2006 006 289.2

(51) Int. Cl. A61L 9/18

(52) U.S. Cl. 422/22; 422/186

(57) ABSTRACT

The invention relates to a device for generating excited and/or ionized particles in a plasma from a process gas, which comprises a generator for generating an electromagnetic wave, a waveguide, and a gas discharge chamber with a gas discharge space in which the excited and/or ionized particles are formed, and comprising a dielectric in which the gas discharge space is formed, the gas discharge chamber being arranged inside the waveguide. In order to be able to use the largest possible microwave powers while achieving a long service life, the dielectric forms an end base from which side walls branch off so as to form the gas discharge space. The electromagnetic wave can also be coupled into the end base.

The invention relates to a device for generating excited and/or ionized particles in a plasma from a process gas, which comprises a generator for generating an electromagnetic wave, a waveguide, and a gas discharge chamber with a gas discharge space, the excited and/or ionized particles being formed in the gas discharge space and the gas discharge chamber comprising a dielectric in which the gas discharge space is formed, the gas discharge chamber being arranged inside the waveguide. The invention also relates to a method for generating excited and/or ionized particles in a plasma from a process gas, in which an electromagnetic wave is generated and is coupled into a dielectric of a gas discharge chamber, there being formed in the dielectric a gas discharge space which comprises a gas inlet and a gas outlet for supplying or removing process gas, and the gas discharge chamber being arranged inside a waveguide.

High-power plasma devices are basically known from the prior art. They are used for example as external plasma sources, as what are known as “remote plasma sources”, for cleaning coating and etching chambers, the plasma being generated in a separate space in order to then convey the excited gas into a reaction chamber through a pipe or other suitable supply means. A further use of the high-power plasma devices lies for example in integrating the devices directly into the coating or etching chamber. It should be noted in this case that, in contrast to remote plasma sources, the excited gas is uniformly distributed over a certain solid angle in the reaction chamber to achieve the desired results.

High-frequency plasma devices which are very efficient are particularly suitable for use in etching and coating processes for semi-conductor components and products from the micromechanics sector. Special plasma devices are required in this case with the process gases being broken down in the smallest space by high-frequency electromagnetic waves and the fractions of which gases are excited further. By using high-power plasma devices etching gases, such as NF₃, CF₄, C₂F₆, SF₆, O₂, etc. are virtually completely broken down into their constituents and as a result are particularly environmentally compatible. As a rule microwaves are used as the electromagnetic waves. By concentrating the microwave energy on the smallest space the materials of the gas discharge chamber are exposed to particularly high thermal loads, with the inner surfaces of the discharge chamber simultaneously being exposed to chemical attack which, as is known, increases exponentially with the temperature of the materials.

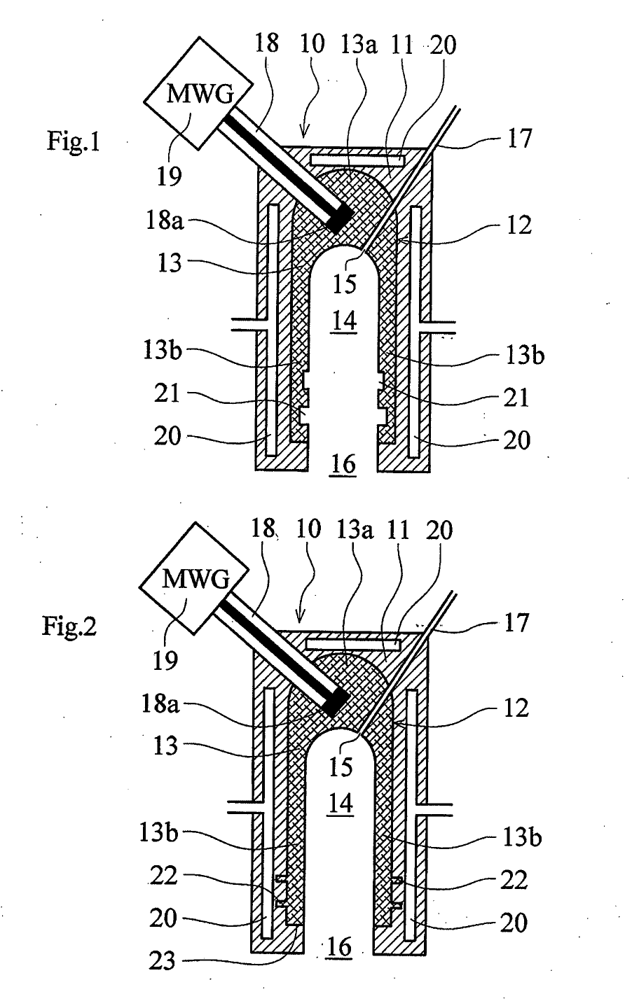

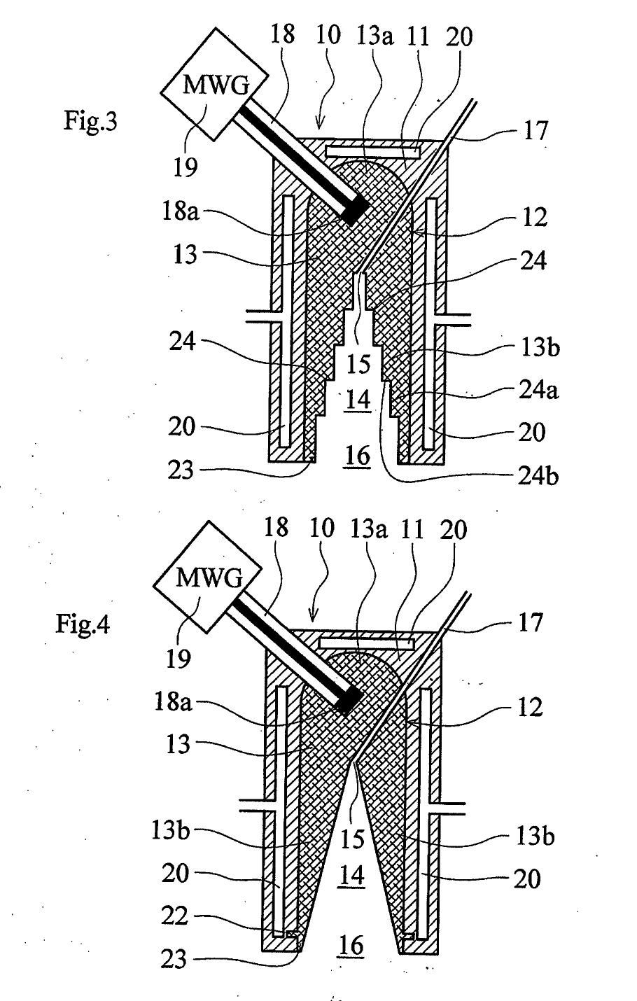

A known high-power plasma device is disclosed for example in JP 07029889 A in which the discharge chamber is arranged in a section of a waveguide, into the end of which, remote from the discharge chamber, a microwave generated by a microwave generator is coupled (see FIG. 4 of JP 07029889 A). With such a device effective cooling of the plasma zone or the discharge chamber is not possible since the microwave has to penetrate into the discharge chamber in the region of the waveguide, and this would be prevented for example by enveloping of the discharge chamber by means of a water cooling device. Complete enveloping of the discharge chamber by a water cooling system is not possible either in the case of high-power plasma devices according to FIGS. 1 and 3 of JP 07029889 A in which the microwave is introduced into the discharge chamber by means of a coaxial conductor system, the internal conductor extending almost over the entire length of the gas discharge chamber. As a result sometimes high thermal loading of the discharge chambers is unavoidable. High mechanical stressing of the discharge chambers, which are usually made from brittle ceramic or glass, occur as a consequence of the non-uniform thermal loadings. Cracks occur in the discharge chambers with higher microwave powers, so the above-described devices may only be used with very restricted power. The anticipated chemical attack on the highly heated parts of the discharge chambers is also considerable.

It is therefore the object of the present invention to disclose a device and a method for generating excited and/or ionized particles in a plasma which can be used with high microwave powers and in which the gas discharge chamber also has a long service life. The object is achieved by a device as claimed in the preamble of claim 1, in which the dielectric forms an end base from which side walls, which also consist of dielectrics, extend so as to form the gas discharge space. The device is also constructed in such a way that electromagnetic waves may be coupled into the end base.

The device according to the invention therefore comprises a gas discharge chamber which in turn comprises a dielectric, for example a ceramic, and a cavity, the gas discharge space, formed in the dielectric. The plasma is generated in the gas discharge space. The gas discharge chamber of the present invention is accordingly constructed such that the dielectric forms an end base. This means that at one end face of the gas discharge chamber the dielectric forms a base or bottom. The base can basically have any desired or expedient shape and consists of the dielectric. Side walls which also consist of dielectrics, branch off this base. The base and the side walls together form a cavity which is used as a gas discharge space. The side walls are therefore expediently circumferential in other words. Moreover in the present invention the electromagnetic waves which are generated by the generator are coupled into the end base of the gas discharge chamber. In contrast to the prior art, in the present invention the electromagnetic wave is therefore coupled or lead into the gas discharge chamber only at certain points and in a locally limited manner. The electromagnetic wave then spreads from the end base through the remainder of the gas discharge chamber, i.e. the side walls made from dielectrics and the gas discharge space in which the required gases are located which are excited by the microwave.

The specific embodiment of the present invention, and in particular the only point-wise coupling of the electromagnetic waves into the gas discharge chamber, makes it possible for the gas discharge chamber to comprise a substantially all-over cooling system to dissipate the heat produced in the discharge space as uniformly as possible to the cooling liquid, for example water. This ensures that the uniform introduction of heat generated in the plasma zone can also be removed uniformly by the cooling system and therefore the mechanical stresses in the dielectric, for example a ceramic or glass body, are minimized. An all-over cooling system also keeps the temperature of the discharge chamber as low as possible, so chemical attack on the chamber material is minimized. A further advantage of the invention is that the discharge space of the excitation and discharge chamber can be constructed in such a way that uniform thermal loading of the discharge chamber is achieved, minimizing mechanical stresses. Overall therefore the productivity of high-power plasma devices, in particular of those which are used in semi-conductor production systems, is increased by the present invention and the environmental compatibility of the processes is improved.

In the device according to the invention the electromagnetic wave, in particular a microwave with the conventional and officially permitted frequencies of 915 MHz, 2.45 GHz or 5.8 GHz, is advantageously coupled into the gas discharge chamber by means of a coupling pin, which is part of a coaxial conductor, through which the wave is guided from the microwave generator to the gas discharge chamber. Coupling or decoupling of microwave energy from a coaxial system into a waveguide system by means of a coupling pin is basically generally known. In the device according to the invention the arrangement of the coupling pins is selected such that all of the microwave energy may be coupled into the gas discharge chamber without some of the energy being reflected. It should be noted in this connection that the propagation velocity of the wave in the dielectric decreases at ε^{1/2} (root of relative dielectric constant) and consequently the dimensions of the coupling pins and their spacing from the reflection planes must be adjusted accordingly. If for example aluminum oxide with a relative dielectric constant of approx. 9 is used as the dielectric, the propagation velocity of the microwave in the dielectric is reduced to a third of the value in air or under vacuum and by appropriate dimensioning of the dielectric and the pin coupling is adjusted accordingly, so the waves are not reflected.

The coupling pins are also expediently dimensioned and fitted into the dielectric of the gas discharge chamber such that there is no impedance jump during the transition of the coaxial conductor to the dielectric of the gas discharge chamber and consequently all of the energy is coupled into the dielectric, without reflection losses occurring. It is particularly advantageous if by appropriate dimensioning of the coupling pin in the region of the dielectric of the gas discharge chamber and leading through into the waveguide, the waveguide is constructed as an electromagnetic oscillating circuit, so the microwave can be fed into the gas discharge chamber particularly effectively. This is achieved for example in that, with respect to its diameter and its length, the coupling pin in the region of the dielectric and waveguide lead-through is constructed so it acts as an oscillating circuit at the given microwave frequency. Consequently a wide variety of device operating conditions, such as pressure difference of 3 powers of ten, and also different process gases now only have a negligible effect on the reflected power of the device.

It is also advantageous for the end of the coupling pin to be fitted into the dielectric so as to be directly adjacent, without there being a gap between coupling pin and dielectric. Consequently the microwave can be fed into the dielectric particularly effectively and without reflection. The heat generated in the coaxial conductor by surface currents may also be dissipated into the dielectric via the coupling pin. This embodiment is particularly suitable for devices with high microwave powers. A further advantage of coupling of the microwaves by way of the coupling pin lies in the fact that, despite a wide variety of device operating conditions with respect to power and pressure, no subsequent adjustments are required.

In addition to capacitive coupling of the microwave energy into the dielectric by means of the coupling pin, inductive coupling by means of a coil is possible. This method is particularly effective with lower frequencies. The microwave can, moreover, also be supplied to the dielectric by waveguide supply lines, in particular in the case of very high frequencies.

In a preferred embodiment of the invention the gas discharge chamber is constructed in such a way that it substantially fills the waveguide. This is taken to mean that the gas discharge chamber is dimensioned such that the interior of the waveguide is substantially completely occupied by the gas discharge chamber. With its outer surface the dielectric therefore adjoins the inner surface of the waveguide and the gas discharge space is in turn formed inside the dielectric. This embodiment is advantageous since on the one hand a compact arrangement may be produced which saves space and on the other hand all of the energy of the microwave remains concentrated on the gas discharge chamber, where it is then consumed in the gas discharge space. The waveguide is conventionally made of metal and is preferably, in particular in this embodiment, constructed as a heat sink, i.e. a cooling system, in particular a water cooling system, is provided in the waveguide. It surrounds the gas discharge chamber from its end base through to the gas outlet. Since the microwave is only coupled into the gas discharge chamber at certain points and otherwise only the relatively small and locally limited inlet for the process gas runs into the gas discharge chamber, the cooling system can adjoin the surface of the gas discharge chamber over a large area and thus an optimum cooling result may be achieved.

The gas discharge chamber is expediently constructed in such a way that provided at the end of the gas discharge space, which opposes the end base, is the gas outlet for the gas discharge space. It is also expedient to provide the gas inlet at the end of the gas discharge space which adjoins the end base. This means that uniform propagation of the plasma over the entire discharge space is established. The thermal loads, viewed over the entire gas discharge chamber, are kept relatively constant thereby, and this in turn contributes to the prevention of damage in the dielectric. It is also ensured that all of the process gas introduced into the gas discharge space is captured by the microwaves and thus a high level of efficiency is established.

The gas discharge chamber is advantageously symmetrical with respect to the longitudinal axis of the waveguide. This embodiment contributes to uniform distribution of the thermal load and simplifies production of the device according to the invention.

The end base is advantageously constructed as a solid, cylindrical or hemispherical body. The inner shape of the waveguide should be adapted accordingly, so it advantageously rests directly on the end base. This results in an advantageous shape for forming the discharge space in cooperation with the side walls, and the microwaves coupled into the end base can be distributed even more uniformly through the entire gas discharge chamber.

In a preferred embodiment of the invention the side walls of the gas discharge chamber comprise at least one cross-sectional taper. This cross-sectional taper is particularly preferably circumferential. As a result of this at least one purposeful cross-sectional taper or reduction in the cross-section of the dielectric, which is particularly preferably provided in the region of the gas outlet, the microwave coupled into the gas discharge chamber can issue into the gas discharge space from the dielectric in the region of the cross-sectional taper in an augmented manner, so a discharge maximum is prevented at the end of the gas discharge space or the gas discharge chamber, i.e. in the region of the gas outlet. Such a discharge maximum could lead to damage to the gas discharge chamber at this location.

A plurality of cross-sectional tapers are advantageously provided, it being particularly advantageous to provide the reduction in dielectric cross-section from the gas inlet to the gas outlet gradually since the microwave energy fed into the end base at the end face of the gas discharge chamber can be gradually supplied to the gas discharge space as a result. Particularly uniform and, as a result, advantageous distribution of the microwave energy can be attained in this connection if the size of the taper increases in the direction of the gas outlet, i.e. in the region of the taper the dielectric cross-section decreases in the direction of the gas outlet.

The at least one cross-sectional taper is expediently in the form of a circumferential recess and in particular a circumferential annular groove. The annular groove can for example comprise a U-shaped cross-sectional profile and is preferably formed on the inner side of the dielectric.

In a further preferred embodiment of the invention the side walls of the gas discharge chamber are constructed such that their cross-section continually tapers in the direction of the gas outlet. This means that their cross-section is continually reduced from the start of the side walls at the end base to their end at the gas outlet. This continual reduction in the cross-section can be provided with a constant degree of reduction or with different degrees of reduction in certain sections. The degree of cross-sectional taper is preferably constant. This may be achieved for example by a conical formation of the discharge space, with the outer sides of the side walls being formed parallel to each other. Consequently the microwave uniformly exits the tapering dielectric cross-section, so the process gas is uniformly excited over the entire conical space. It is also advantageous in this embodiment for the steepness of the cone to predefine the solid angle at which the excited gases issue from the gas discharge space if the gas outlet is constructed such that it runs over the entire width of the cone base. The solid angle can be pre-defined such that the excited process gases are distributed optimally uniformly over a respective workpiece.

In a further preferred embodiment of the invention the side walls of the discharge chamber comprise at least one, in particular circumferential, projection. The projection protrudes from the side of the side walls facing the waveguide and the extent of the, as a rule, U-shaped cross-section of the projection beyond the side wall corresponds to half the wavelength of the electromagnetic wave (λ/2) in the dielectric. In the case of a U-shaped cross-section the extent is therefore composed of the added-together lengths of the two U-legs and the connecting piece between the U-legs. In principle the cross-section of the projection can also have any other desired shape, wherein care should always be taken that the cross-sectional extent corresponds to λ/2. The at least one projection acts as a blocking element for microwaves and is used to limit the propagation of the microwaves. The at least one projection is therefore expediently provided at the gas outlet, and thus in the gas flow direction, at the end of the gas discharge chamber. Propagation of the microwave at the end of the gas discharge chamber, i.e. in the region of the gas outlet, can be limited thereby. This prevents the microwaves from being able to issue from the gas discharge chamber and enter into the processing or reaction space connected downstream, and in which a workpiece is to be arranged for processing by the excited process gases, which would have an adverse effect on the processing operation. The at least one projection is particularly preferably constructed as a circumferential bead and in particular as a dielectric ring with a relatively small width. The dielectric ring is placed onto the dielectric side walls. As a result of the fact that the at least one, advantageously U-shaped, projection has a cross-sectional extent of half the wavelength of the microwave, a positive half-wave is generated on one side of the projection and a negative half-wave on the other, which half-waves overlie each other and thus compensate to zero. This means that the electromagnetic waves are prevented from reaching the end of the discharge chamber opposing the end base and being able to cause damage there. With particularly high microwave powers it is particularly preferred for blocking elements to be combined with cross-sectional tapers of the side walls, so the energy is reliably consumed in the gas discharge space up to the gas outlet. The at least one projection can likewise be provided in a device according to the invention of which the side walls are constructed with a cross-section that continually tapers in the direction of the gas outlet.

In a further preferred embodiment at least one, in particular circumferential, shoulder is provided on the inner sides of the side walls. A plurality of shoulders is particularly preferably arranged one after the other, so a pyramid-like graduation of the gas discharge space results. The graduations are preferably provided such that the gas discharge space widens in the direction of the gas outlet. The length of the shoulders corresponds to a distance of the electromagnetic wave of λ/4 in the dielectric. A large number of maxima are produced at the shoulders thereby which are reflected from the preceding electromagnetic wave and the succeeding electromagnetic wave reflected at the shoulders of the side walls, it being possible to purposefully improve the effectiveness of the gas discharge in the maxima and it also being possible to distribute the microwave energy throughout the gas discharge space at uniform intervals. With a discharge space graduated in a pyramid-like manner the solid angle, at which the gases issue from this space, can be predefined and corresponds to the slope of the pyramid. For this purpose the gas outlet should be formed such that it extends over the entire base of the graduated pyramid. The solid angle can therefore be adapted to the dimensions of the workpieces to be treated. Instead of λ/4 the length of the shoulders may also be λ/4+nλ, where n=1, 2, 3, etc.

The waveguide is preferably substantially cuboidal, cylindrical, elliptical or conical. If the wave has the shape of a circular cylinder the diameter of the waveguide in the region of the end base is expediently selected such that it is greater than the cut-off wavelength of the electromagnetic wave and thus propagation of the electromagnetic wave is possible in at least the basic mode. The field configuration of the electromagnetic wave in the cylindrical waveguides is best illustrated in cylindrical coordinates. In cylindrical coordinates the solution to the wave equation provides the Bessel function. In the region of the discharge space of the gas discharge chamber the diameter of the waveguide should also be selected such that it is greater than the cut-off wavelength of the microwave. If the waveguide is cuboidal, the width of the waveguide should expediently be predefined such that it is greater than λ/2 of the electromagnetic wave. Appropriate selection of the diameter of the round waveguide or the width of the cuboidal waveguide allows the formation of an advantageous number of electromagnetic wave modes.

The waveguide may also be constructed such that it has different shapes in certain regions. According to a further preferred embodiment the waveguide is cylindrical in the region of the end base and widens conically in the further region of the gas discharge space to allow a gas discharge chamber with a combustion space or gas discharge space with a particularly large solid angle. As a result large-area workpieces, such as semi-conductor wafers with a diameter of 300 mm, can be processed very uniformly with excited gases. In a particularly advantageous embodiment of the invention the gas discharge space of the gas discharge chamber is also conical and the cross-section of the side walls of the dielectric tapers uniformly in the direction of the gas outlet, so the microwave can issue uniformly. At the end of the gas discharge space there is provided a microwave blocking element in the form a circumferential annular bead. It is also possible in this embodiment to provide shoulders on the inner side of the side walls. The cross-section of the waveguide surrounding the gas discharge chamber is advantageously round, elliptical or rectangular.

According to the respective requirements with respect to the size of the workpieces to be processed and the microwave power required, excitation chambers of different sizes can be built and the correspondingly suitable microwave frequencies (for example 915 MHz, 2.45 GHz or 5.8 GHz) selected therefor. This means for example that, with an identical design, the 915 MHz device is approx. six times larger than the 5.8 GHz device.

In a further preferred embodiment the gas discharge chamber is fitted in the waveguide by means of interference fit. Consequently the heat produced during gas discharge may be particularly effectively onwardly conveyed to the cooling liquid by means of the interference fit of the cooling jacket on the gas discharge chamber, thus allowing very effective cooling of the gas discharge chamber.

Some typical application examples of high-power plasma devices constructed according to the present invention will be given below.

Remote Plasma Sources

Processes for Cleaning Coating Chambers and Etching Chambers:

Microwave power: 2 to 30 kW, preferably 2 to 6 kW

Frequency: 2.45 GHz or 915 MHz

Pressure: 0.5 to 5 torr

Gases: NF₃, C₂F₆+O₂, SF₆+O₂, Cl₂+NF₃

Plasma Sources with Conical Gas Egress and Relatively Large Solid Angle

Stripping of Photoresists and Etching of Workpieces:

Microwave power: 0.5 to 30 kW, preferably 0.5 to 4 kW

Frequency: 2.45 GHz or 915 MHz

Pressure: 0.1 to 5 torr

Gases: O₂, N₂, forming gas, NF₃, CF₄

Activation and Cleaning of Surfaces:

Microwave power: 0.5 to 30 kW, preferably 0.5 to 4 kW

Frequency: 2.45 GHz

Pressure: 0.05 to 5 torr

Gases: O₂, N₂, H₂, forming gas, CF₄, Ar

The object is also achieved by a method for generating excited and/or ionized particles in a plasma from a process gas in which an electromagnetic wave is generated and is coupled into a dielectric of a gas discharge chamber, there being formed in the dielectric a gas discharge chamber which comprises a gas inlet and a gas outlet for supplying or removing process gas. The electromagnetic wave is also coupled into an end base of the dielectric, the gas discharge space being arranged between the end base and the gas outlet. By appropriate configuration of the dielectric the energy of the electromagnetic wave coupled into the gas discharge chamber in the end base is also preferably consumed in the method in the gas discharge space up until the gas outlet is reached.

The invention will be described in more detail hereinafter with reference to embodiments illustrated in the drawings, in which schematically:

FIG. 1 shows a device for generating excited and/or ionized particles in a plasma with cross-sectional tapers in the side walls,

FIG. 2 shows the device from FIG. 1 in which, instead of the cross-sectional tapers, projections protruding outwardly from the side walls are formed,

FIG. 3 shows a device for generating excited and/or ionized particles in a plasma with stepped shoulders arranged one behind the other,

FIG. 4 shows a device for generating excited and/or ionized particles in a plasma of which the side walls have a continually tapering cross-section,

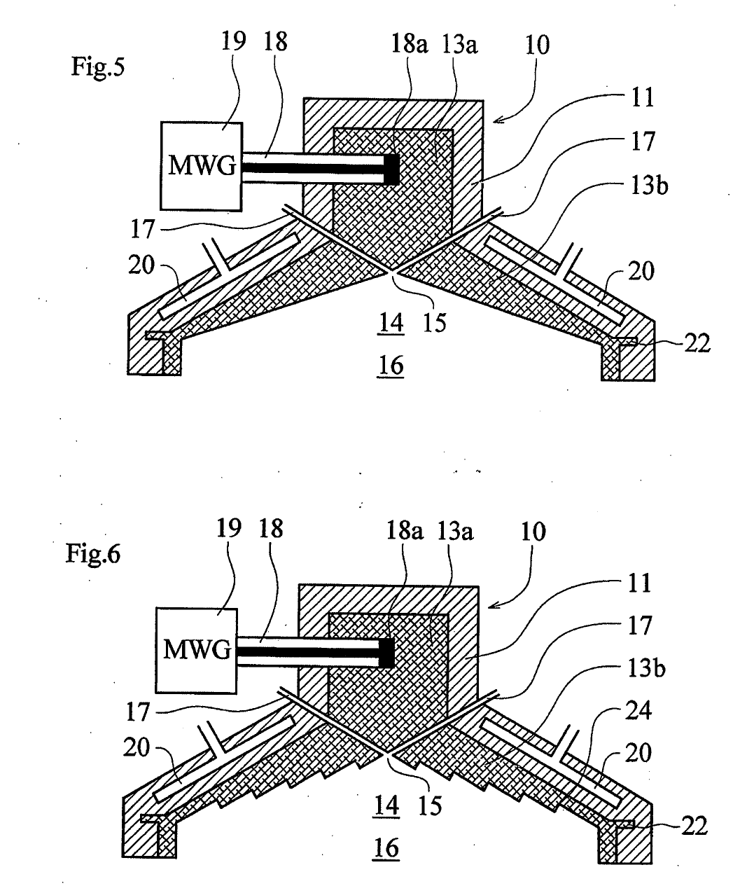

FIG. 5 shows a device for generating excited and/or ionized particles in a plasma with a partially conical waveguide,

FIG. 6 shows the device from FIG. 5 with stepped shoulders arranged one behind the other being provided on the inner side of the side walls,

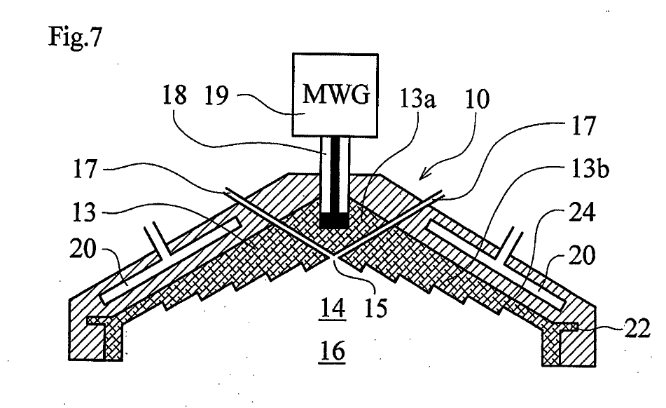

FIG. 7 shows the device from FIG. 6, with the entire waveguide being conical,

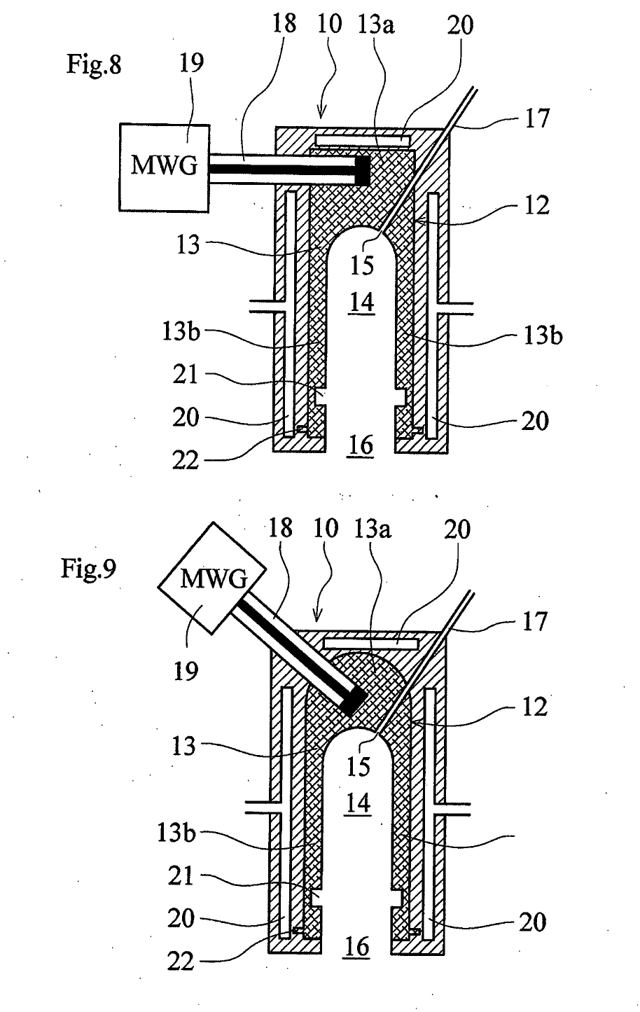

FIG. 8 shows a device for generating excited and/or ionized particles in a plasma in which the coaxial conductor couples the microwaves laterally into the end base,

FIG. 9 shows a device for generating excited and/or ionized particles in a plasma in which the coaxial conductor couples the electromagnetic wave obliquely from above into the end base,

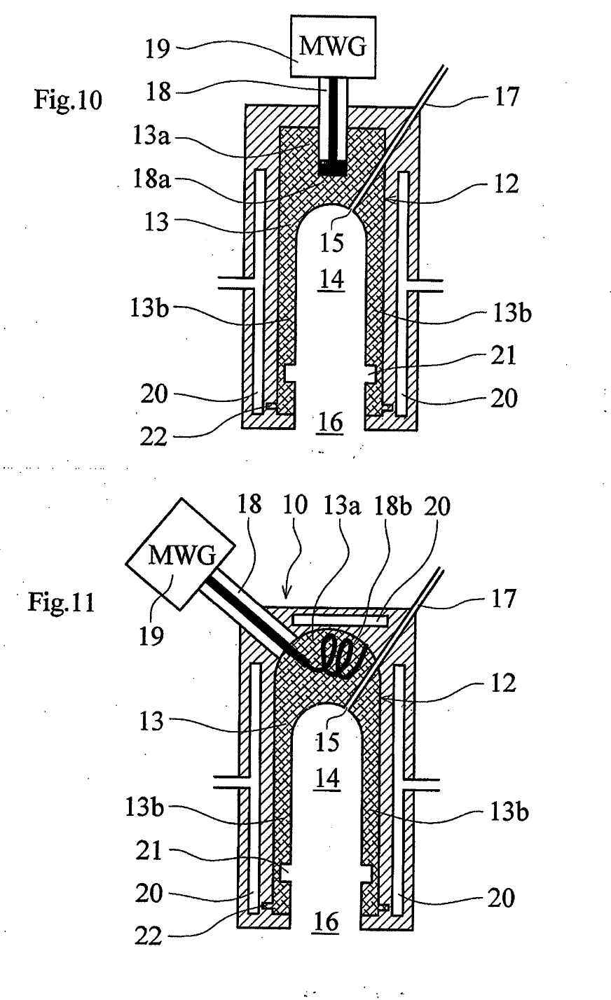

FIG. 10 shows the device from FIG. 8, with the coupling being performed from above,

FIG. 11 shows the device from FIG. 9, with the microwave being inductively coupled-in,

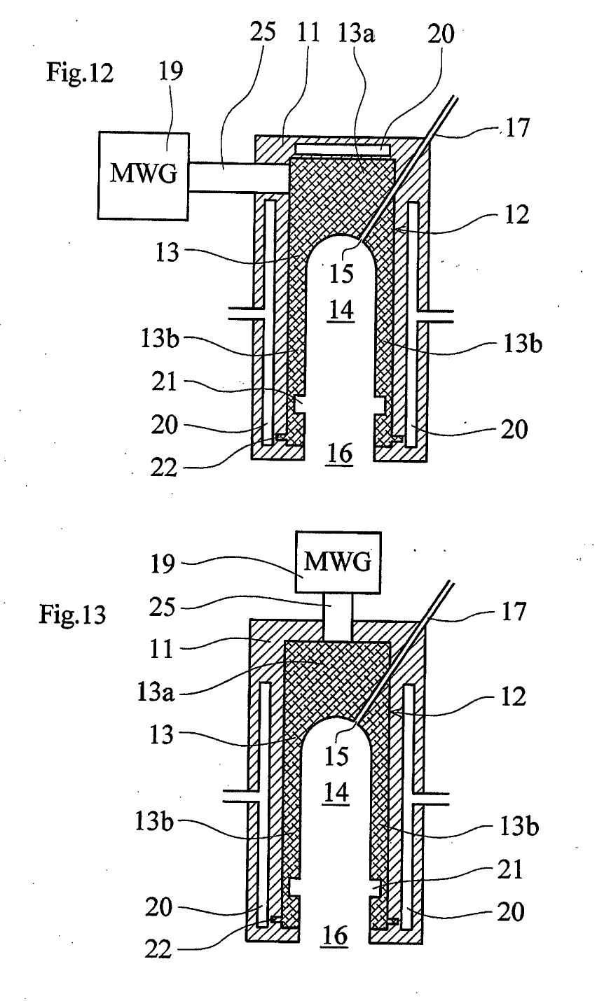

FIG. 12 shows the device according to FIG. 8, with the microwave being coupled-in from the side by means of a waveguide supply line,

FIG. 13 shows the device from FIG. 12, with coupling being performed from above.

In the various embodiments described hereinafter identical reference numerals are used for identical parts. All illustrations shown in the figures are longitudinal sections through the device according to the invention.

FIG. 1 shows a device 10 for generating excited and/or ionized particles in a plasma from a process gas. The plasma-generating device 10 comprises a circular cylindrical waveguide 11 which is produced from a suitable material, in particular a metal. The waveguide 11 comprises a likewise circular cylindrical, and at its front, closed end, hemispherical cavity in which the gas discharge chamber 12 is arranged. The gas discharge chamber 12 in turn consists of a body of dielectric 13 and a gas discharge space 14 formed in the dielectrics. The dielectric 13 rests with all of its outer surface on the inner side of the waveguide 11. The dielectric 13 consists of an end base 13a and side walls 13b which branch off from the end base 13a and form a cavity or gas discharge space 14. The end base 13a rests on a closed end of the waveguide 11. The side walls 13b are circumferential. The side walls 13b are also formed with a constant width in the circumferential direction and the inner an outer sides of the side walls 13b are substantially parallel to each other. Both waveguide 11 and dielectric 13 have an open free end, with the dimensions of the openings each being substantially identical and both openings being arranged congruently one on top of the other. These openings form the gas outlet 16. The diameter of the gas outlet 16 substantially corresponds to the diameter of the gas discharge space 14. It is arranged so as to oppose the end base 13a. The gas inlet 15 is in turn formed directly on the end base 13a and connected to a gas supply 17 via which the gas is introduced into the gas discharge space 14.

An opening is provided in the waveguide 11 through which a coaxial conductor 18 runs which ends in the end base 13a of the dielectric 13. At its end the coaxial conductor 18 has a coupling pin 18a which is fitted into the end base 13a in such a way that it rests on the dielectric on all sides. This coaxial conductor 18 is connected to a microwave generator 19. The generated microwaves are coupled from the microwave generator 19 into the end base 13a via the coaxial conductor 18 and by means of the coupling pin 18a thereof. From the base the microwaves propagate through the entire gas discharge chamber 12. The coaxial conductor 18 is introduced into the plasma-generating device from oblique top left. In the waveguide 11, in both its end region and in the side walls, there are cooling lines 20, along which cooling liquid, in particular water, flows to cool the gas discharge chamber 11. The cooling lines 20 substantially cover the entire surface region of the gas discharge chamber 12 and are locally interrupted by only the coaxial conductor 18 and the gas supply 17. Two circumferential annular grooves 21 are provided on the inner side of the side walls 13b of the dielectric 13. The annular grooves 21 have a rectangular cross-section and, viewed in the longitudinal direction, are arranged in the region of the side walls 13b facing the gas outlet 16. The upper annular groove has a shallower depth than the lower one, i.e. the dielectric is less thick in the region of the lower annular groove than in the region of the upper annular groove.

The plasma generating device 10 illustrated in FIG. 2, in contrast to that in FIG. 1, does not have a cross-sectional taper or annular grooves 21. Instead two circumferential projections, which are constructed as circumferential annular beads 22, are provided on the dielectric 13. The annular beads 22 are provided on the lower end of the side walls 13b in the region of the gas outlet 16. They project from the surface of the outer side of the side walls 13b. Corresponding recesses are provided in the waveguide 11 which enclose the annular beads 22 with interlocking fit. The annular beads 22 have a U-shaped cross-section and stand substantially orthogonally on the side walls 13b. Their cross-section is dimensioned such that the extent of the bead cross-section corresponds to a distance of the electromagnetic wave of λ/2, so superimposition of the positive and negative half-waves is compensated to zero and the electrical wave is prevented from reaching the end of the discharge chamber 23. The embodiments of the present invention illustrated in FIGS. 1 and 2 are particularly advantageously suitable for what are known as “remote plasma sources”.

In the case of the plasma generating device 10 shown in FIG. 3 shoulders 24 are provided on the inner side of the side walls 13b. The shoulders 24 are consecutively arranged from the end base 13a through to the gas outlet 16 and are circumferential, so pyramid-shaped or stepped graduations of the gas discharge chamber 14 result, with the gas discharge space 14 widening toward the gas outlet 16. A shoulder in each case comprises a vertical and a horizontal flank 24a, 24b which are substantially orthogonal to each other. The length of the vertical flank 243a is λ/4 of the distance of the electromagnetic wave. Consequently the energy of the microwaves introduced into the end base 13a is consumed up to the open end 23 of the gas discharge chamber 12.

In the case of the device 10 illustrated in FIG. 4 the gas discharge space 14 is conical with the apex of the cone abutting the end base 13a and the base of the cone forming the gas outlet 16. The conical construction of the gas discharge space 14 with vertical and mutually parallel alignment of the outer sides of the side walls 13b results in continuous tapering of the side walls 13b from the end base 13a in the direction of the end of the gas discharge chamber 23. A blocking element 22 constructed as an annular bead is provided at the end of the gas discharge chamber 23. In both FIGS. 3 and 4 the excited gases in each case exit the device 10 at a specific solid angle which is predefined by the elevation of the cone or the graduated pyramids. Consequently it is expedient with these devices to arrange the reaction chamber with the workpiece to be processed so as to directly adjoin the gas outlet and to arrange the workpiece under the gas outlet 16 in such a way that it is completely covered by the excited gases.

FIG. 5 shows a plasma generating device 10 in which, in the region of the end base 13a, the waveguide 11 is circular cylindrical-shaped and in the remaining region, i.e. in the region which surrounds the end walls 13b, is conical. The side walls 13b, according to the device in FIG. 4, are also constructed in such a way that their cross-section tapers in the direction of the gas outlet 16. The conical construction of the waveguide and gas discharge space 14 means that, with the device 10 in FIG. 5, a particularly large solid angle may be attained at which the excited gas exits the discharge space 14. Circumferential annular beads 22 acting as blocking elements are also provided in the region of the gas outlet 16.

The device illustrated in FIG. 6 is similar to that in FIG. 5. One difference lies in stepped shoulders 24 being provided on the inner side of the side walls 13b. Regular zones of particularly high plasma density are produced thereby, and this is particularly advantageous with respect to the process results that can be anticipated. To prevent propagation of the microwave beyond the discharge chamber a circumferential annular bead 22 is provided in the region of the gas outlet 16 in this device as well. In the devices 10 illustrated in FIGS. 5 and 6 the coaxial conductor, or the coupling pins 18a provided thereon, is laterally introduced into the end base 13a. With the embodiment of the end base 13a care should be taken that a shape is chosen which allows reflection-free coupling of the microwave energy.

The device in FIG. 7 is similar to that in FIG. 6, wherein in contrast thereto the waveguide 11 and the dielectric 13 are conical in their entireties. This means that the end base 13a forms the apex region of the cone of the dielectric 13. Two gas supply lines 17, which each run obliquely from above into the device 10 and end in the upper end point of the gas discharge space 14, are also disposed in the present case. Moreover, in contrast to FIG. 6 the coaxial conductor 18 is introduced vertically from above into the end base 13a.

FIGS. 8 and 10 each show a device 10 in which the end base 13a is constructed as a circular cylindrical body which consists entirely of the dielectric. The side walls 13b adjoin thereto so as to form the gas discharge space 14. The side walls 13b have an annular groove 21 and an annular bead 22. The upper end region of the gas discharge space 14 is hemispherical and with its end face 13a adjoin the end base 13a. In FIG. 8 the coaxial conductor 18 is introduced laterally, and in FIG. 10 vertically from above, into the end base 13a.

In FIGS. 9 and 11, in contrast to the devices in FIGS. 8 and 10, the end base 13a is hemispherical. The hemispherical construction of the end base of the gas discharge chamber is particularly suitable for coupling of the microwave obliquely from above since the reflected power is particularly low in all applications of the device. A coil 18b is provided in FIG. 11 instead of a coupling pin. Coupling of the microwave energy into the end base 13a in the device in FIG. 11 is therefore performed inductively. In both devices the coaxial conductor 18 is obliquely introduced into the device 10 from the top left in each case.

In the devices in FIGS. 12 and 13 the microwave is coupled from the microwave generating device 19 into the end base 13a via a respective waveguide supply line 25. The waveguide supply lines 25 are constructed in such a way that they pass through a recess in the waveguide 11 and end at the outer edge region of the end base 13a. In the device in FIG. 12 the waveguide supply line 25 is introduced laterally, and in the device in FIG. 13 vertically from above, into the device 10.

1. A device for generating excited and/or ionized particles in a plasma from a process gas, comprising:
a generator for generating an electromagnetic wave;
a waveguide;
a gas discharge chamber with a gas discharge space in which the excited and/or ionized particles are formed; and
a dielectric in which the gas discharge space is formed, the gas discharge chamber being arranged inside the waveguide, wherein the dielectric forms an end base from which side walls extend so as to form the gas discharge space, and wherein the electromagnetic wave can be coupled into the end base.

2. The device as claimed in claim 1, wherein the gas discharge chamber substantially fills the waveguide.

3. The device as claimed in claim 1 comprising a gas inlet and a gas outlet for supplying or removing process gas into or from the gas discharge space, characterized in that the gas outlet is provided at the end of the gas discharge space opposing the end base.

4. The device as claimed in claim 3, wherein the gas inlet is provided at the end of the gas discharge space facing the end base.

5. The device as claimed in claim 1, wherein the gas discharge chamber is formed symmetrically with respect to the longitudinal axis of the waveguide.

6. The device as claimed in claim 1, wherein the end base is constructed as a cylindrical or hemispherical body.

7. The device as claimed in claim 1, wherein the side walls of the gas discharge chamber comprise at least one, in particular circumferential, cross-sectional taper.

8. The device as claimed in claim 7, comprising a gas inlet and a gas outlet for supplying or removing process gas into or from the gas discharge space, characterized in that the at least one cross-sectional taper is provided in the region of the gas outlet.

9. The device as claimed in claim 7, comprising a gas inlet and a gas outlet for supplying or removing process gas into or from the gas discharge space, characterized in that a plurality of cross-sectional tapers is provided, the size of the tapers increasing in the direction of the gas outlet.

10. The device as claimed in claim 7 wherein the at least one cross-sectional taper is constructed in the form of a circumferential annular groove.

11. The device as claimed in claim 1, further comprising a gas inlet and a gas outlet for supplying or removing process gas into or from the gas discharge space, wherein the side walls of the gas discharge chamber are constructed in such a way that their cross-section continually tapers in the direction of the gas outlet.

12. The device as claimed in claim 1, wherein the side walls of the gas discharge chamber comprise at least one, in particular circumferential, projection which protrudes from the side of the side walls facing the waveguide and of which the cross-sectional extent corresponds to half the wavelength of the electromagnetic shaft.

13. The device as claimed in claim 12, comprising a gas inlet and a gas outlet for supplying or removing process gas into or from the gas discharge space, wherein the at least one projection is provided in the region of the gas outlet.

14. The device as claimed in claim 12 wherein the at least one projection is constructed as a circumferential bead.

15. The device as claimed in claim 1, wherein provided at the side of the side walls facing the gas discharge space is at least one, in particular circumferential, shoulder, of which the length corresponds to a quarter of the wavelength of the electromagnetic wave.

16. The device as claimed in claim 15, comprising a gas inlet and a gas outlet for supplying or removing process gas into or from the gas discharge space, characterized in that the gas outlet is provided at the end of the gas discharge space opposing the end base, and in that a plurality of circumferential shoulders are arranged in a stepped manner one behind the other such that the gas discharge space widens in the gas outlet direction.

17. The device as claimed in claim 1, wherein the waveguide is substantially cuboidal, cylindrical or conical.

18. The device as claimed in claim 17, wherein the waveguide is cuboidal, the width of the waveguide being greater than half the wavelength of the electromagnetic wave.

19. The device as claimed in claim 17, wherein the waveguide is constructed as a circular cylinder, the diameter of the cylinder being greater than the cut-off wavelength of the electromagnetic wave.

20. The device as claimed in claim 1, wherein the waveguide is differently shaped in certain sections.

21. The device as claimed in claim 20, wherein the waveguide is cylindrical in the region of the end base and conical in the region of the gas discharge space.

22. The device as claimed in claim 1, wherein the gas discharge chamber is fitted in the waveguide with interference fit.

23. The device as claimed in claim 1, wherein provided on the waveguide is a cooling system which envelops the gas discharge chamber over a large area.

24. The device as claimed in claim 1, wherein the electromagnetic wave can be coupled into the end base by means of a coupling pin or a coupling coil of a coaxial conductor.

25. The device as claimed in claim 24, wherein at its end the coupling pin comprises a junction which is arranged directly adjacent to the dielectric.

26. The device as claimed in claim 1, wherein the electromagnetic wave can be coupled into the end base by means of a waveguide supply line.

27. A method for generating excited and/or ionized particles in a plasma from a process gas in which an electromagnetic wave is generated and is coupled into a dielectric of a gas discharge chamber, comprising:
forming in the dielectric a gas discharge space which comprises a gas inlet and a gas outlet for supplying or removing process gas, with the gas discharge chamber being arranged inside a waveguide, wherein the electromagnetic wave is coupled into an end base of the dielectric, the gas discharge space is arranged between the end base and the gas outlet.

28. The method as claimed in claim 27, wherein as a result of suitable configuration of the dielectric the energy of the electromagnetic wave coupled into the gas discharge chamber in the end base is consumed up until the gas outlet is reached.
